(19) United States
(12) Patent Application Publication
(10) Pub. No.: US 2021/0061800 A1
(43) Pub. Date: Mar. 4, 2021

(54) COMPOUNDS FOR TREATING EYE DISEASES AND METHODS THEREOF

(71) Applicant: Agency for Science, Technology and Research, Singapore (SG)

(72) Inventors: Surana UTTAM, Singapore (SG); Hong Hwa LIM, Singapore (SG); Chandra VERMA, Singapore (SG); Srinivasaraghavan KANNAN, Singapore (SG); Kong Peng LAM, Singapore (SG); Gireedhar VENKATACHALAM, Singapore (SG); Shi Qin Vanessa NG, Singapore (SG)

(21) Appl. No.: 16/958,546

(22) PCT Filed: Dec. 31, 2018
(86) PCT No.: PCT/SG2018/050642

§ 371 Date: Jun. 26, 2020

(30) Foreign Application Priority Data
Dec. 29, 2017 (SG) 10201710908U

(51) Int. Cl. C07D 471/04; A61K 9/00; C07K 16/22; C07K 16/28; C07D 257/04

(52) U.S. Cl. CPC C07D 471/04; A61K 9/0019; A61K 9/0048; C07D 257/04; C07K 16/22; C07K 16/2863; A61K 9/0053

(57) ABSTRACT

A compound of formula (I) or a salt, solvate or prodrug thereof:

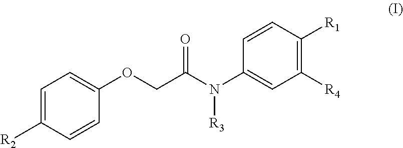

wherein R₁ and R₄ are independently selected from C₁-C₅ alkyl, C₁-C₅ haloalkyl, C₁-C₅ alkenyl, C₁-C₅ haloalkenyl, C₁-C₅ alkynyl, C₁-C₅ haloalkynyl, C₁-C₅ alkoxy or C₁-C₅ haloalkoxy;
R₂ is an optionally substituted heteroaryl selected from optionally substituted tetrazolyl or optionally substituted imidazopyridinyl; and
R₃ is selected from H or optionally substituted alkyl.

### FIELD

The present invention relates to compounds and methods for treating eye diseases, for example macular degeneration (MD) and in particular age-related macular degeneration (AMD). The present invention also relates to methods for treating diabetic retinopathy.

### BACKGROUND

Macular degeneration (MD) is a medical condition that usually occurs in the elderly (also referred to as age-related macular degeneration (AMD)) and may result in a loss of vision in the macula due to retinal damage. MD and AMD are complex disorders that involve molecular derangement in or near Bruch's membrane, the multilaminar sandwich of extracellular matrix that lies between the retinal pigment epithelium and the anastomotic blood supply of the outer retina known as choriocapillaris. This derangement can lead to the growth of new blood vessels (commonly referred to as choroidal neovascularization) from the choriocapillaris through Bruch's membrane and into the subretinal pigment epithelial or subretinal space. This is also known as wet MD or wet AMD. This neovascular complication can be so damaging to the structure and function of the retina it results in blindness attributable to the disease. For example, these new blood vessels may bleed and leak fluid, causing the macula to bulge or lift up from its normally flat position, thus distorting or destroying central vision. Under these circumstances, vision loss may be rapid and severe. Approximately 10-15% of the cases of MD (or AMD) are the “wet” (exudative) type, and about 90% of these cases results in a loss of vision.

Currently known treatments for MD or AMD involving choroidal neovascularization include anti-VEGF (Vascular Endothelial Growth Factor) antibodies that cause regression of abnormal blood vessel growth and improvement of vision when injected directly into the vitreous humour of the eye. Examples of these antibodies include the monoclonal antibodies to VEGF such as ranibizumab (marketed as “Lucentis”), bevacizumab (marketed as “Avastin”), pegatanib (marketed as “Macugen”) and aflibercept (marketed as “Eylea”). Treatments involving the use of these drugs are often expensive and often not effacious. In this regard, a patient is requires to get an injection to the eye once per month, with each injection costing about USD 1,800 (Eylea) to about USD 2,000 (Lucentis). Such a procedure is invasive and 20-30% of patients do not respond to the treatment. The cause for this non-responsiveness is unknown.

Diabetic retinopathy is a medical condition in which damage occurs to the retina due to diabetes and is a leading cause of blindness. It affects up to 80% of people who have had diabetes for 20 years or more. Each year in the United States, diabetic retinopathy accounts for 12% of all new cases of blindness. It is also the leading cause of blindness for people aged 20 to 64 years. Diabetic retinopathy is the result of damage to the small blood vessels and neurons of the retina. The earliest changes detected in the retina in diabetes leading to diabetic retinopathy include a narrowing of the retinal arteries associated with reduced retinal blood flow; dysfunction of the neurons of the inner retina, followed in later stages by changes in the function of the outer retina, associated with subtle changes in visual function; dysfunction of the blood-retinal barrier, which protects the retina from many substances in the blood (including toxins and immune cells), leading to the leaking of blood constituents into the retinal neuropile. The basement membrane of the retinal blood vessels subsequently thickens, the capillaries degenerate, leading to loss of blood flow, progressive ischemia, microscopic aneurysms (balloon-like structures jutting out from the capillary walls which recruit inflammatory cells), and advanced dysfunction and degeneration of the neurons and glial cells of the retina.

Thus far, the best way of preventing the onset and delaying the progression of diabetic retinopathy is to monitor it vigilantly and to achieve optimal glycemic control. Intravitreal medication, such as triamcinolone acetonide, is available but requires repeated injections to the eye. Another side effect is that vision may become more blurred and/or with the appearance of black spots after administering triamcinolone acetonide as the drug precipitates out in the eye.

There is therefore a clear need to identify alternative beneficial therapies for the treatment of MD or AMD, in particular wet MD or AMD, for instance, non-antibody based therapies for the treatment of MD or AMD. There is also a clear need to identify alternative beneficial therapies for the treatment of diabetic retinopathy.

### SUMMARY OF THE INVENTION

The present invention is predicated on the discovery that the particular diphenyl oxyacetamide based compounds disclosed herein inhibit angiogenesis, and therefore may be useful in the treatment of eye diseases such as macular degeneration (MD) and/or aged related macular degeneration (AMD), in particular wet MD or wet AMD, or diabetic retinopathy.

In one aspect, the present invention provides a compound of formula (I) or a salt, solvate or prodrug thereof:

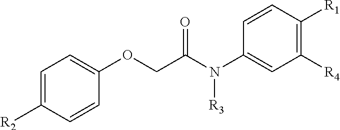

wherein R₁ and R₄ are independently selected from C₁-C₅ alkyl, C₁-C₅ haloalkyl, C₁-C₅ alkenyl, C₁-C₅ haloalkenyl, C₁-C₅ alkynyl, C₁-C₅ haloalkynyl, C₁-C₅ alkoxy or C₁-C₅ haloalkoxy;
R₂ is an optionally substituted heteroaryl selected from optionally substituted tetrazolyl or optionally substituted imidazopyridinyl; and
R₃ is selected from H or optionally substituted alkyl.

In an embodiment, the present invention provides a pharmaceutical composition comprising an effective amount of a compound of formula (I) or a pharmaceutically acceptable salt, solvate or prodrug thereof:

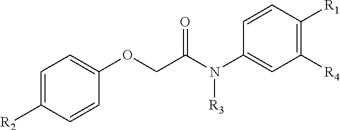

optionally in combination with a pharmaceutically acceptable carrier, excipient or diluent.

In an embodiment, the present invention provides a pharmaceutical combination comprising an effective amount of a compound of formula (I) or a pharmaceutically acceptable salt, solvate or prodrug thereof:

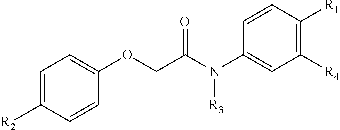

and an effective amount of a second therapeutic agent.

In another aspect, the present invention provides a method for treating macular degeneration (MD) in a patient in need thereof, the method comprising administering to the patient a therapeutically effective amount of a compound of formula (I) or a pharmaceutically acceptable salt, solvate or prodrug thereof:

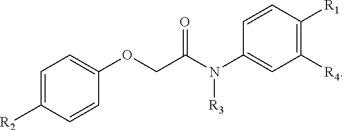

In certain embodiments, the present invention provides a method for treating wet macular degeneration (MD) in a patient in need thereof, the method comprising administering to the patient a therapeutically effective amount of a compound of formula (I) or a pharmaceutically acceptable salt, solvate or prodrug thereof:

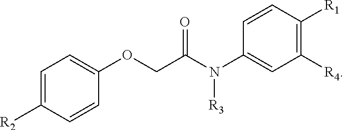

In another aspect, the present invention provides a use of a compound of formula (I) or a pharmaceutically acceptable salt, solvate or prodrug thereof:

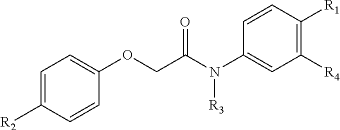

in the manufacture of a medicament for treating macular degeneration (MD) in a patient in need thereof.

In certain embodiments, the present invention provides a use of a compound of formula (I) or a pharmaceutically acceptable salt, solvate or prodrug thereof:

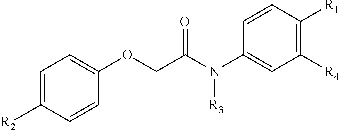

in the manufacture of a medicament for treating wet macular degeneration (MD) in a patient in need thereof.

In another aspect, the present invention provides a compound of formula (I) or a pharmaceutically acceptable salt, solvate or prodrug thereof:

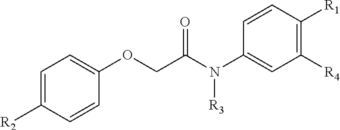

for use in treating macular degeneration (MD) in a patient in need thereof.

In certain embodiments, the present invention provides a compound of formula (I) or a pharmaceutically acceptable salt, solvate or prodrug thereof:

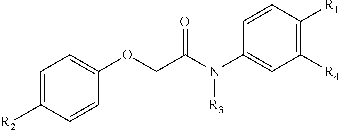

for use in treating wet macular degeneration (MD) in a patient in need thereof.

In another aspect, the present invention provides a method for treating diabetic retinopathy in a patient in need thereof, the method comprising administering to the patient a therapeutically effective amount of a compound of formula (I) or a pharmaceutically acceptable salt, solvate or prodrug thereof:

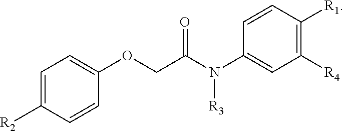

In another aspect, the present invention provides a use of a compound of formula (I) or a pharmaceutically acceptable salt, solvate or prodrug thereof:

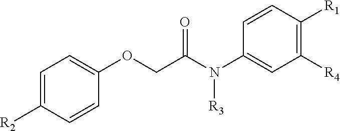

in the manufacture of a medicament for treating diabetic retinopathy in a patient in need thereof.

In another aspect, the present invention provides a compound of formula (I) or a pharmaceutically acceptable salt, solvate or prodrug thereof:

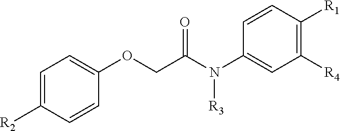

for use in treating diabetic retinopathy in a patient in need thereof.

### BRIEF DESCRIPTION OF THE FIGURES

FIG. 1 illustrates an NMR result of Ex 1.

FIG. 2 illustrates an NMR result of Ex 2.

FIG. 3 illustrates an NMR result of Ex 3.

FIG. 4 depicts a graph of OD reading relative to time (hrs) in relation to Ex 1 (a compound of the present invention), Gleevac (Imatinib), DMSO and raff/gal (blank). The efficacy of Ex 1 was compared to Gleevec (a well-known inhibitor of PDGFR) at 10 μM concentration for its capacity to prevent PDGFR-induced death of humanized yeast cells.

FIG. 5 illustrates a Western-blot analysis result of Ex 1 (a compound of the present invention), in relation to inhibit PDGFRβ signaling in HEK293 cells expressing PDGFRβ.

FIG. 6 illustrates a work flow for the synthesis of compounds of formula (I) based on in silico modelling and simulation to improve the efficacy toward PDGFRβ.

FIG. 7 illustrates a Western-blot analysis result of Ex 2 and Ex 3 (compounds of the present invention) to inhibit PDGFRβ signaling in BaF3 cells expressing PDGFRβ.

FIG. 8 illustrates IC₅₀ results of Ex 2 and Ex 3 (compounds of the present invention) using in vitro kinase assay and kinome array. The IC₅₀ result of Gleevac (Imatinib) is shown as a comparison.

FIG. 9 illustrates IC₅₀ profiles of Ex 2 and Ex 3 (compounds of the present invention) against PDGFRα, PDGFRβ and VEGFR2 using in vitro kinase assay.

FIG. 10 illustrates the efficacy of the Ex 2 and Ex 3 (compounds of the present invention) in an ex vivo organoid model of angiogenesis.

FIG. 11 illustrates the efficacy of Ex 3 (a compound of the present invention) in Laser CNV mouse model for wet-AMD.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like. As used herein, C₁-C₅ alkyl refers to an alkyl group having 1 to 5 carbon atoms.

“Alkylene” refers to divalent alkyl groups preferably having from 1 to 10 carbon atoms and more preferably 1 to 6 carbon atoms. Examples of such alkylene groups include methylene (—CH₂—), ethylene (—CH₂CH₂—), and the propylene isomers (e.g., —CH₂CH₂CH₂— and —CH(CH₃)CH₂—), and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like. As used herein, C₁-C₅ alkylenyl refers to an alkylenyl group having 1 to 5 carbon atoms.

“Alkynyl” refers to alkynyl groups preferably having from 2 to 10 carbon atoms and more preferably 2 to 6 carbon atoms and having at least 1, and preferably from 1-2, carbon to carbon, triple bonds. Examples of alkynyl groups include ethynyl (—C≡CH), propargyl (—CH₂C≡CH), pent-2-ynyl (—CH₂C≡CCH₂—CH₃), and the like. As used herein, C₁-C₅ alkynyl refers to an alkynyl group having 1 to 5 carbon atoms.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like. As used herein, C₁-C₅ alkoxy refers to an alkoxy group having 1 to 5 carbon atoms.

“Halo” or “halogen” refers to fluoro, chloro, bromo and iodo.

“Haloalkyl” refers to an alkyl group wherein the alkyl group is substituted by one or more halo group as described above. The terms “haloalkenyl”, “haloalkynyl” and “haloalkoxy” are likewise defined.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Hückel criteria for aromaticity (ie. contains 4n+2π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

In this specification, the term “macular degeneration” or “MD” is intended to include age-related macular degeneration (AMD), but does not exclude macular degeneration in patients who are not elderly. Accordingly, AMD and MD as referred to herein may be used interchangeably. MD is a disease that affects a special layer of cells in the eye called the retinal pigment epithelium. This layer of cells is underneath the retina. The retinal pigment epithelium (RPE) is like a wall or barrier and is responsible for passing oxygen, sugar and other essentials up to the retina and moving waste products down to the blood vessels underneath (these vessels are called the choroid). The RPE also acts as a barrier between the choroid and the retina. When RPE cells die, the retinal cells above them also die, leading to patches of ‘missing’ retina. This is commonly called geographic atrophy or “dry” MD, which is a slow form of the disease that causes a gradual loss of vision. “Wet” macular degeneration occurs when the RPE cells fail to stop choroidal blood vessels from growing under the retina. This growth is called choroidal neovascularisation or CNV. The rapidly growing vessels are fragile with leaky walls and they ooze fluid and blood under the retina. This leads to scarring and severe loss of central vision, which if left untreated, becomes permanent. In the context of the present invention it will be appreciated that the term “macular degeneration” particularly refers to “wet” MD also known as neovascular or exudative AMD.

As used herein, the term “diabetic retinopathy” refers to a microvascular complication of diabetes. This complication can occur in the eye. Accordingly, “diabetic retinopathy” is intended to include all categories and classification, for example the earlier stage of nonproliferative diabetic retinopathy (NPDR) and the advanced stage of proliferative diabetic retinopathy (PDR) associated with abnormal blood vessel growth. Diabetic macular edema (DME) is also included within its scope. DME is a manifestation of diabetic retinopathy that occurs across all severity levels of both NPDR and PDR and represents the most common cause of vision loss in patients. DME arises from diabetes-induced breakdown of the blood-retinal barrier (BRB), with consequent vascular leakage of fluid and circulating proteins into the neural retina. The extravasation of fluid into the neural retina leads to abnormal retinal thickening and often cystoid edema of the macula.

The present invention is based on the discovery that diphenyl oxyacetamide based compounds of formula (I), or a pharmaceutically acceptable salt, solvate or prodrug thereof:

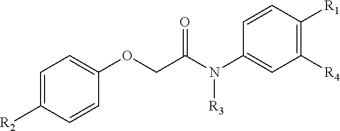

inhibit angiogenesis, and therefore may be useful in the treatment of macular degeneration (MD) and/or aged related macular degeneration (AMD), in particular wet MD or wet AMD.

In an embodiment, R₁ and R₄ are independently selected from C₁-C₅ alkyl, C₁-C₅ haloalkyl, C₁-C₅ alkenyl, C₁-C₅ haloalkenyl, C₁-C₅ alkynyl, C₁-C₅ haloalkynyl, C₁-C₅ alkoxy or C₁-C₅ haloalkoxy. In another embodiment, R₁ and R₄ are independently selected from C₁-C₅ alkyl, C₁-C₅ haloalkyl, C₁-C₅ alkenyl, C₁-C₅ haloalkenyl, C₁-C₅ alkoxy or C₁-C₅ haloalkoxy. In another embodiment, R₁ and R₄ are independently selected from C₁-C₅ alkyl, C₁-C₅ haloalkyl, C₁-C₅ alkenyl or C₁-C₅ haloalkenyl. In another embodiment, R₁ and R₄ are independently selected from C₁-C₅ alkyl or C₁-C₅ haloalkyl.

In an embodiment, at least one of R₁ or R₄ is C₁-C₅ alkyl. In another embodiment, at least one of R₁ or R₄ is methyl. In another embodiment, at least one of R₁ or R₄ is ethyl. In another embodiment, at least one of R₁ or R₄ is n-propyl. In another embodiment, at least one of R₁ or R₄ is iso-propyl. In another embodiment, at least one of R₁ or R₄ is n-butyl. In another embodiment, at least one of R₁ or R₄ is sec-butyl. In another embodiment, at least one of R₁ or R₄ is isobutyl. In another embodiment, at least one of R₁ or R₄ is tert-butyl. In another embodiment, at least one of R₁ or R₄ is n-pentyl. In another embodiment, at least one of R₁ or R₄ is 2-methylbutan-2-yl. In another embodiment, at least one of R₁ or R₄ is 2,2-dimethylpropyl. In another embodiment, at least one of R₁ or R₄ is 3-methylbutyl. In another embodiment, at least one of R₁ or R₄ is sec-pentyl. In another embodiment, at least one of R₁ or R₄ is 3-pentyl. In another embodiment, at least one of R₁ or R₄ is sec-isopentyl. In another embodiment, at least one of R₁ or R₄ is 2-methylbutyl.

In an embodiment, both R₁ and R₄ are C₁-C₅ alkyl. In another embodiment, both R₁ and R₄ are methyl. In another embodiment, both R₁ and R₄ are ethyl. In another embodiment, both R₁ and R₄ are n-propyl. In another embodiment, both R₁ and R₄ are iso-propyl. In another embodiment, both R₁ and R₄ are n-butyl. In another embodiment, both R₁ and R₄ are sec-butyl. In another embodiment, both R₁ and R₄ are isobutyl. In another embodiment, both R₁ and R₄ are tert-butyl. In another embodiment, both R₁ and R₄ are n-pentyl. In another embodiment, both R₁ and R₄ are 2-methylbutan-2-yl. In another embodiment, both R₁ and R₄ are 2,2-dimethylpropyl. In another embodiment, both R₁ and R₄ are 3-methylbutyl. In another embodiment, both R₁ and R₄ are sec-pentyl. In another embodiment, both R₁ and R₄ are 3-pentyl. In another embodiment, both R₁ and R₄ are sec-isopentyl. In another embodiment, both R₁ and R₄ are 2-methylbutyl.

In an embodiment, at least one of R₁ or R₄ is C₁-C₅ is haloalkyl. In another embodiment, R₄ is C₁-C₅ is haloalkyl. In another embodiment, R₄ is halomethyl. In another embodiment, R₄ is dihalomethyl. In another embodiment, R₄ is trihalomethyl. In another embodiment, R₄ is haloethyl. In another embodiment, R₄ is dihaloethyl. In another embodiment, R₄ is trihaloethyl. In another embodiment, R₄ is tetrahaloethyl. In another embodiment, R₄ is pentahaloethyl. In another embodiment, R₄ is fluoromethyl. In another embodiment, R₄ is difluoromethyl. In another embodiment, R₄ is trifluoromethyl. In another embodiment, R₄ is fluoroethyl. In another embodiment, R₄ is difluoroethyl. In another embodiment, R₄ is trifluoroethyl. In another embodiment, R₄ is tetrafluoroethyl. In another embodiment, R₄ is pentafluoroethyl.

In an embodiment, R₂ is an optionally substituted heteroaryl. In another embodiment, R₂ is optionally substituted tetrazolyl. In another embodiment, R₂ is optionally substituted imidazopyridinyl. In another embodiment, R₂ is selected from optionally substituted tetrazolyl or optionally substituted imidazopyridinyl. In another embodiment, R₂ is selected from tetrazolyl or imidazopyridinyl.

In an embodiment, when R₂ is an optionally substituted heteroaryl, the optionally substituted group is selected from hydroxyl, acyl, alkyl, alkoxy, alkenyl, alkenyloxy, alkynyl, alkynyloxy, amino, aminoacyl, carboxyl, acylamino, cyano, halogen, nitro, phosphono, sulfo, phosphorylamino, phosphinyl, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents. In another embodiment, the optionally substituted group is selected from hydroxyl, acyl, alkyl, alkoxy, alkenyl, alkenyloxy, alkynyl, alkynyloxy, amino, aminoacyl, carboxyl, acylamino, cyano, halogen, nitro, oxyacyl, trihalomethyl, pentafluoroethyl, trifluoromethoxy, difluoromethoxy, trifluoroethenyl, mono- and di-alkylamino, mono- and di-(substituted alkyl)amino.

In an embodiment, R₃ is selected from H or optionally substituted alkyl. In another embodiment, R₃ is H. In another embodiment, R₃ is optionally substituted alkyl. In another embodiment, R₃ is methyl. In another embodiment, R₃ is ethyl. In another embodiment, R₃ is n-propyl. In another embodiment, R₃ is iso-propyl. In another embodiment, R₃ is n-butyl. In another embodiment, R₃ is sec-butyl. In another embodiment, R₃ is isobutyl. In another embodiment, R₃ is tert-butyl. In another embodiment, R₃ is n-pentyl. In another embodiment, R₃ is 2-methylbutan-2-yl. In another embodiment, R₃ is 2,2-dimethylpropyl. In another embodiment, R₃ is 3-methylbutyl. In another embodiment, R₃ is sec-pentyl. In another embodiment, R₃ is 3-pentyl. In another embodiment, R₃ is sec-isopentyl. In another embodiment, R₃ is 2-methylbutyl.

In an embodiment, R₁ is C₁-C₅ alkyl, R₂ is optionally substituted heteroaryl, R₃ is selected from H or optionally substituted alkyl and R₄ is C₁-C₅ alkyl. In another embodiment, R₁ is C₁-C₅ alkyl, R₂ is optionally substituted heteroaryl, R₃ is H and R₄ is C₁-C₅ alkyl. In another embodiment, R₁ is C₁-C₅ alkyl, R₂ is optionally substituted heteroaryl, R₃ is optionally substituted alkyl and R₄ is C₁-C₅ alkyl.

In another embodiment, R₁ is C₁-C₅ alkyl, R₂ is optionally substituted heteroaryl, R₃ is selected from H or optionally substituted alkyl and R₄ is C₁-C₅ haloalkyl. In another embodiment, R₁ is C₁-C₅ alkyl, R₂ is optionally substituted heteroaryl, R₃ is H and R₄ is C₁-C₅ haloalkyl. In another embodiment, R₁ is C₁-C₅ alkyl, R₂ is optionally substituted heteroaryl, R₃ is optionally substituted alkyl and R₄ is C₁-C₅ haloalkyl.

In another embodiment, R₁ is C₁-C₅ haloalkyl, R₂ is optionally substituted heteroaryl, R₃ is selected from H or optionally substituted alkyl and R₄ is C₁-C₅ haloalkyl. In another embodiment, R₁ is C₁-C₅ haloalkyl, R₂ is optionally substituted heteroaryl, R₃ is H and R₄ is C₁-C₅ haloalkyl. In another embodiment, R₁ is C₁-C₅ haloalkyl, R₂ is optionally substituted heteroaryl, R₃ is optionally substituted alkyl and R₄ is C₁-C₅ haloalkyl.

In another embodiment, R₁ is methyl, R₂ is optionally substituted heteroaryl, R₃ is selected from H or optionally substituted alkyl and R₄ is methyl. In another embodiment, R₁ is methyl, R₂ is optionally substituted heteroaryl, R₃ is selected from H or optionally substituted alkyl and R₄ is trifluoromethyl. In another embodiment, R₁ is methyl, R₂ is optionally substituted heteroaryl, R₃ is H and R₄ is methyl. In another embodiment, R₁ is methyl, R₂ is optionally substituted heteroaryl, R₃ is optionally substituted alkyl and R₄ is methyl. In another embodiment, R₁ is methyl, R₂ is optionally substituted heteroaryl, R₃ is H and R₄ is trifluoromethyl. In another embodiment, R₁ is methyl, R₂ is optionally substituted heteroaryl, R₃ is optionally substituted alkyl and R₄ is trifluoromethyl.

In another embodiment, R₁ is methyl, R₂ is optionally substituted tetrazolyl, R₃ is selected from H or optionally substituted alkyl and R₄ is methyl. In another embodiment, R₁ is methyl, R₂ is optionally substituted tetrazolyl, R₃ is selected from H or optionally substituted alkyl and R₄ is trifluoromethyl. In another embodiment, R₁ is methyl, R₂ is optionally substituted imidazopyridinyl, R₃ is selected from H or optionally substituted alkyl and R₄ is methyl. In another embodiment, R₁ is methyl, R₂ is optionally substituted imidazopyridinyl, R₃ is selected from H or optionally substituted alkyl and R₄ is trifluoromethyl.

Accordingly, in an embodiment, compound of formula (I) may be selected from the following:

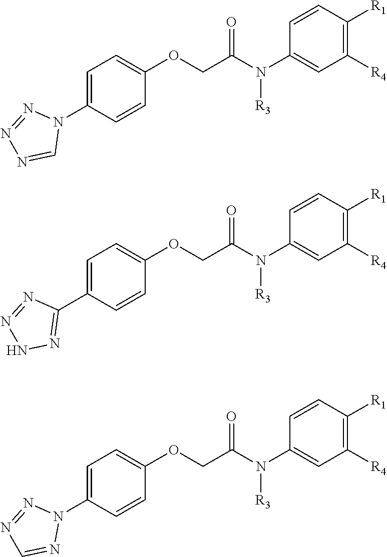

or a pharmaceutically acceptable salt, solvate or prodrug thereof. In another embodiment, the compounds as shown above may be optionally substituted at tetrazolyl.

In another embodiment, compound of formula (I) may be selected from the following:

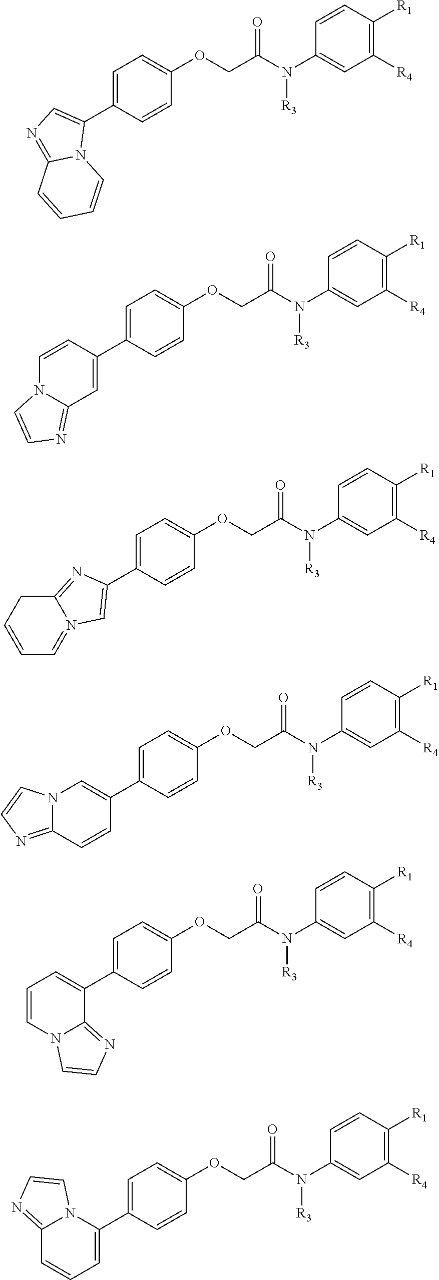

or a pharmaceutically acceptable salt, solvate or prodrug thereof. In another embodiment, the compounds as shown above may be optionally substituted at imidazopyridinyl.

In certain embodiments, compound of formula (I) may be selected from the following:

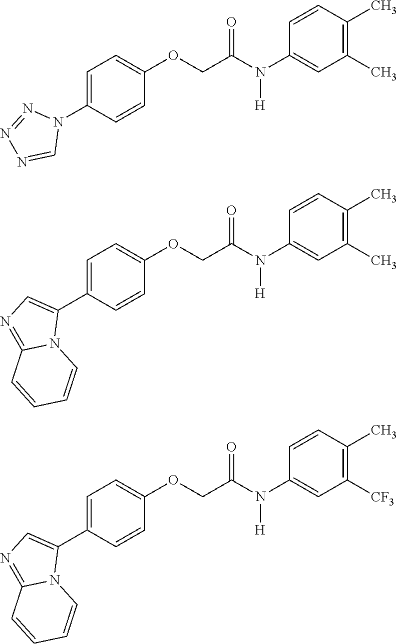 [SMILES: Cc1ccc(NC(=O)COc2ccc(-n3cnnn3)cc2)cc1C ; Cc1ccc(NC(=O)COc2ccc(-c3cnc4ccccn34)cc2)cc1C ; Cc1ccc(NC(=O)COc2ccc(-c3cnc4ccccn34)cc2)cc1C(F)(F)F]

or a pharmaceutically acceptable salt, solvate or prodrug thereof.

In an embodiment, the present invention provides a pharmaceutical composition comprising an effective amount of a compound of formula (I) or a pharmaceutically acceptable salt, solvate or prodrug thereof:

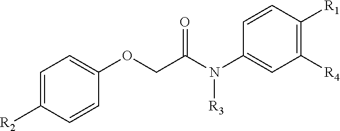

optionally in combination with a pharmaceutically acceptable carrier, excipient or diluent.

As used herein, the term “composition” relates to a combination of the different chemical substances to produce a final product. The final product may be a single final product, and may also include an active ingredient. Accordingly, it will be appreciated that a composition with one or more active ingredient(s) is also within the scope and spirit of the invention. Such a composition may be administered by any route, and can be in any dosage form and amount.

In wet AMD and diabetic retinopathy, VEGFα is believed to play a significant role in the formation of blood vessels that grow abnormally and leak beneath the macula. The constant exposure of endothelial cells to pro-angiogenic factors, such as VEGFα, result in the formation of immature, semi-differentiated and fragile blood vessels which have a tendency to leak and bleed. Without wanting to be bound by theory, the present invention is based on the discovery that a compound of formula (I) as defined herein displays high selectivity towards the receptor tyrosine kinases (RTKs) PDGFRα, PDGFRβ and VEGFR2, the three main RTKs responsible for abnormal blood vessel growth in the context of MD. These receptor tyrosine kinases are high affinity cell surface receptors for polypeptide growth factors such as VEGFα. Accordingly, it is postulated that the compounds of the present invention may exhibit a wider therapeutic window than compounds or agents that do not distinguish between “diseased” and normal cells. This selectivity means the compounds of formula (I), as well as pharmaceutically acceptable salt, solvate or prodrug thereof, may be particularly well suited for therapeutic application to patients with macular degeneration as they may be able to inhibit proliferation of only “diseased” cells; i.e. with high density of receptor tyrosine kinases. It is believed that the present compounds may be effective in blocking the sprouting of abnormal blood vessel formation, and accordingly be advantageous for treating MD and/or diabetic retinopathy.

The disease pathology of MD and/or diabetic retinopathy can be multi-factorial. In the treatment of MD and/or diabetic retinopathy, different therapies may be combined (i.e. combination therapies). The term “therapeutic agent”, “other therapeutic agent”, “another therapeutic agent”, “second therapeutic agent” and the like, as used herein is intended to include other therapeutic compounds or treatments which may be used in combination with the compound according to the present invention. These second therapeutic agents include, but are not limited to, angiogenesis inhibitors, vascular endothelial growth factor (VEGF) inhibitors, other receptor tyrosine kinase inhibitors, photodynamic therapy, laser photocoagulation, as well as other MD or AMD and/or diabetic retinopathy specific treatments. For example, a compound of formula (I) or a pharmaceutically acceptable salt, solvate or prodrug may be administered in combination with one or more VEGF inhibitors such as avastin, lucentis and/or macugen.

In an embodiment, the present invention provides a pharmaceutical combination comprising an effective amount of a compound of formula (I) or a pharmaceutically acceptable salt, solvate or prodrug thereof:

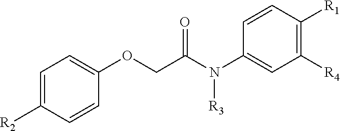

and an effective amount of a second therapeutic agent.

As used herein, the term “combination” relates to the co-administration of the combination partners to a single patient, and are intended to include treatment regimens in which the agents are not necessarily administered by the same route of administration or at the same time. The therapeutic compounds or treatments used in such combination therapies may be administered together with a compound of formula (I) or a pharmaceutically acceptable salt, solvate or prodrug, one after the other, separately in one combined unit dosage or in separate unit dosage forms.

Examples of therapeutic agents for use in combination therapies according to the present invention include VEGF inhibitors such as avastin, lucentis and/or macugen. In an embodiment, the pharmaceutical combination comprises an effective amount of a compound of formula (I) or a pharmaceutically acceptable salt, solvate or prodrug and an effective amount of a VEGF inhibitor. In another embodiment, the pharmaceutical combination comprises an effective amount of a compound of formula (I) or a pharmaceutically acceptable salt, solvate or prodrug and an effective amount of avastin. In another embodiment, the pharmaceutical combination comprises an effective amount of a compound of formula (I) or a pharmaceutically acceptable salt, solvate or prodrug and an effective amount of lucentis. In another embodiment, the pharmaceutical combination comprises an effective amount of a compound of formula (I) or a pharmaceutically acceptable salt, solvate or prodrug and an effective amount of macugen. In another embodiment, the pharmaceutical combination comprises an effective amount of a compound of formula (I) or a pharmaceutically acceptable salt, solvate or prodrug and an effective amount of eylea.

An example of a treatment for use in combination therapies according to the present invention is photodynamic therapy. In an embodiment, a compound of formula (I) or a pharmaceutically acceptable salt, solvate or prodrug is administered to a patient in need thereof in combination with photodynamic therapy (PDT). In this respect, the patient may be subjected to the PDT before, at the same time or after a compound of formula (I) or a pharmaceutically acceptable salt, solvate or prodrug has been administered to the patient. PDT is an art known technique which would be familiar to the skilled person.

In one aspect of the present invention there is provided a method for treating macular degeneration (MD) in a patient in need thereof, the method comprising administering to the patient a therapeutically effective amount of a compound of formula (I) or a pharmaceutically acceptable salt, solvate or prodrug thereof:

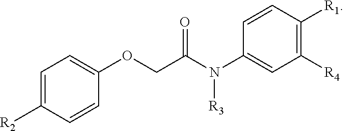

There is also provided a use of a therapeutically effective amount of a compound of formula (I) or a pharmaceutically acceptable salt, solvate or prodrug thereof, in the manufacture of a medicament for treating macular degeneration (MD) in a patient in need thereof.

The present invention also provides a compound of formula (I) or a pharmaceutically acceptable salt, solvate or prodrug thereof for use in treating macular degeneration (MD) in a patient in need thereof.

In an embodiment, the present invention provides a pharmaceutical composition for use in treating macular degeneration (MD) in a patient in need thereof, comprising an effective amount of a compound of formula (I) or a pharmaceutically acceptable salt, solvate or prodrug thereof, and optionally in combination with a pharmaceutically acceptable carrier, excipient or diluent.

In an embodiment, the present invention provides a method for treating macular degeneration (MD) in a patient in need thereof, the method comprising administering to the patient a therapeutically effective amount of a compound of formula (I) or a pharmaceutically acceptable salt, solvate or prodrug thereof, and an effective amount of a second therapeutic agent.

In an embodiment, the present invention also provided a use of a therapeutically effective amount of a compound of formula (I) or a pharmaceutically acceptable salt, solvate or prodrug thereof, and an effective amount of a second therapeutic agent, in the manufacture of a medicament for treating macular degeneration (MD) in a patient in need thereof.

In an embodiment, the present invention also provides an effective amount of a compound of formula (I) or a pharmaceutically acceptable salt, solvate or prodrug thereof, and an effective amount of a second therapeutic agent for use in treating macular degeneration (MD) in a patient in need thereof.

In an embodiment, the present invention provides a pharmaceutical combination for use in treating macular degeneration (MD) in a patient in need thereof, comprising an effective amount of a compound of formula (I) or a pharmaceutically acceptable salt, solvate or prodrug thereof and an effective amount of a second therapeutic agent.

In certain embodiments, the macular degeneration is wet macular degeneration.

In another aspect of the present invention there is provided a method for treating diabetic retinopathy in a patient in need thereof, the method comprising administering to the patient a therapeutically effective amount of a compound of formula (I) or a pharmaceutically acceptable salt, solvate or prodrug thereof:

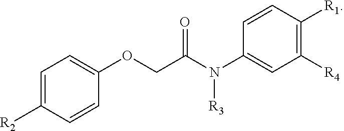

There is also provided a use of a therapeutically effective amount of a compound of formula (I) or a pharmaceutically acceptable salt, solvate or prodrug thereof, in the manufacture of a medicament for treating diabetic retinopathy in a patient in need thereof.

The present invention also provides a compound of formula (I) or a pharmaceutically acceptable salt, solvate or prodrug thereof for use in treating diabetic retinopathy in a patient in need thereof.

In an embodiment, the present invention provides a pharmaceutical composition for use in treating diabetic retinopathy in a patient in need thereof, comprising an effective amount of a compound of formula (I) or a pharmaceutically acceptable salt, solvate or prodrug thereof, and optionally in combination with a pharmaceutically acceptable carrier, excipient or diluent.

In an embodiment, the present invention provides a method for treating diabetic retinopathy in a patient in need thereof, the method comprising administering to the patient a therapeutically effective amount of a compound of formula (I) or a pharmaceutically acceptable salt, solvate or prodrug thereof, and an effective amount of a second therapeutic agent.

In an embodiment, the present invention also provided a use of a therapeutically effective amount of a compound of formula (I) or a pharmaceutically acceptable salt, solvate or prodrug thereof, and an effective amount of a second therapeutic agent, in the manufacture of a medicament for treating diabetic retinopathy in a patient in need thereof.

In an embodiment, the present invention also provides an effective amount of a compound of formula (I) or a pharmaceutically acceptable salt, solvate or prodrug thereof, and an effective amount of a second therapeutic agent for use in treating diabetic retinopathy in a patient in need thereof.

In an embodiment, the present invention provides a pharmaceutical combination for use in treating diabetic retinopathy in a patient in need thereof, comprising an effective amount of a compound of formula (I) or a pharmaceutically acceptable salt, solvate or prodrug thereof and an effective amount of a second therapeutic agent.

In an embodiment, a compound of formula (I) or a pharmaceutically acceptable salt, solvate or prodrug is administered to a patient in need thereof in combination with avastin; or a compound of formula (I) or a pharmaceutically acceptable salt, solvate or prodrug is administered to a patient in need thereof in combination with lucentis; or a compound of formula (I) or a pharmaceutically acceptable salt, solvate or prodrug is administered to a patient in need thereof in combination with macugen. Again, the VEGF inhibitors including avastin, lucentis and/or macugen used in such combination therapies may be administered together, one after the other, separately in one combined unit dosage or in separate unit dosage forms.

The compound of the invention can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any compound that is a prodrug of the compound of formula (I) is also within the scope and spirit of the invention. Thus the compound of the invention can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers).

The compound of the invention may be in crystalline form either as the free compound or as a solvate (e.g. hydrate) and it is intended that both forms are within the scope of the present invention. Methods of solvation are generally known within the art.

The compound of the invention, or a pharmaceutically acceptable salt, solvate or prodrug thereof is administered to the patient in a therapeutically effective amount. As used herein, a therapeutically effective amount is intended to include at least partially attaining the desired effect, or delaying the onset of, or inhibiting the progression of, or halting or reversing altogether the onset or progression of macular degeneration.

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages may lie within the range of about 0.1 ng per kg of body weight to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

Suitable dosage amounts and dosing regimens can be determined by the attending physician and may depend on the severity of the condition as well as the general age, health and weight of the patient to be treated.

The compound of the invention may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

In an embodiment, the compound of formula (I) or a pharmaceutically acceptable salt, solvate or prodrug is injected directly to the eye, and in particular the vitreous of the eye. The compound, composition or combination of the invention can be administered to the vitreous of the eye using any intravitreal or transscleral administration technique. For example, the compound, composition or combination can be administered to the vitreous of the eye by intravitreal injection. Intravitreal injection typically involves administering a compound of the invention or a pharmaceutically acceptable salt, solvate or prodrug in a total amount between 0.1 ng to 10 mg per dose.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants. By way of example, the compound, composition or combination can be dissolved in a pharmaceutically effective carrier and be injected into the vitreous of the eye with a fine gauge hollow bore needle (e.g., 30 gauge, ½ or ⅜ inch needle) using a temporal approach (e.g., about 3 to about 4 mm posterior to the limbus for human eye to avoid damaging the lens).

In an embodiment, a compound of formula (I) or a pharmaceutically acceptable salt, solvate or prodrug thereof may be formulated in a saline solution and injected into the vitreous of the eye. In a preferred embodiment, the compound of formula (I) or a pharmaceutically acceptable salt, solvate or prodrug is a disodium phosphate ester of the compound formula (I), and 0.1 ng to 10 mg of the disodium phosphate ester of the compound of formula (I) is formulated in a 0.9% saline solution and injected directly into the vitreous of the eye.

A person skilled in the art will appreciate that other means for injecting and/or administering the compound, composition or combinations to the vitreous of the eye can also be used. These other means can include, for example, intravitreal medical delivery devices. These devices and methods can include, for example, intravitreal medicine delivery devices, and biodegradable polymer delivery members that are inserted in the eye for long term delivery of medicaments. These devices and methods can further include transscleral delivery devices.

Although intravitreal administration is likely to be a form of administration, the present invention also includes other modes of administration including topical or intravenous administration. For example, solutions or suspensions of the compound, composition or combinations of the invention may be formulated as eye drops, or as a membranous ocular patch, which is applied directly to the surface of the eye. Topical application typically involves administering the compound of the invention in an amount between 0.1 ng and 10 mg.

In another embodiment, the compound of formula (I) or a pharmaceutically acceptable salt, solvate or prodrug is provided on the surface of the eye. The compound may be provided on the surface of the eye as an eye drop, in particular as an eye drop composition or combination. The compound, composition or combinations of the invention can be administered to the surface of the eye using any known administration technique. For example, the compound or combinations can be administered to the surface of the eye by dripping the formulation onto the eye.

The compound, composition or combinations of the invention may also be suitable for intravenous administration. For example, a compound of formula (I) or a pharmaceutically acceptable salt, solvate or prodrug thereof may be administered intravenously at a dose of up to 16 mg/m².

The compound, composition or combinations of the invention may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the compound of formula (I) or a pharmaceutically acceptable salt, solvate or prodrug is orally administerable.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound, composition or combinations of the invention may be suitable for topical administration in the mouth including lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound, composition or combinations of the invention may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The compound, composition or combination of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage composition or combinations are those containing a daily dose or unit, daily sub-dose, as herein above described, or an appropriate fraction thereof, of the active ingredient.

It should be understood that in addition to the active ingredients particularly mentioned above, the composition or combination of this invention may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Those skilled in the art will appreciate that the invention described herein in susceptible to variations and modifications other than those specifically described. It is to be understood that the invention includes all such variations and modifications which fall within the spirit and scope. The invention also includes all of the steps, features, combinations, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

### Identification of Compounds

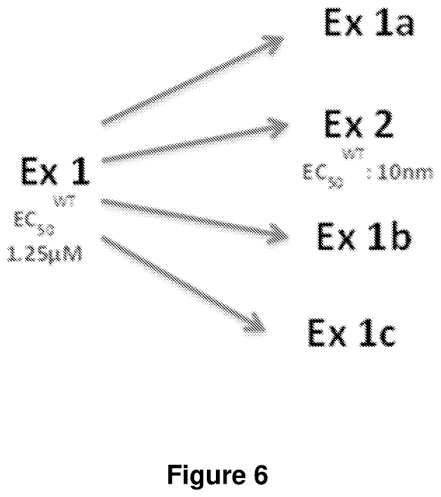

FIG. 6 illustrates a work flow for the synthesis of compound of formula (I) derivatives based on in silico modelling and simulation to improve the efficacy toward PDGFRβ.

Based on in silico modelling and simulation, different derivatives of the diphenyl oxyacetamide based compounds (formula (I)) were synthesized. The potency of the compounds were measured in terms of its ability to inhibit the PDGFRβ-dependent growth of Ba/F3 cells. In particular, Ex 2 and Ex 3 showed marked improvement in its potency against PDGFR-WT with effective concentration-50 (EC50) of 10 nm.

### Synthetic Protocols

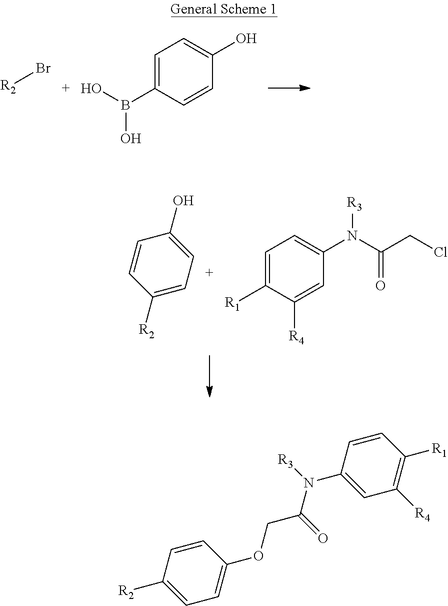

Compounds of the present invention can be synthesized based on the scheme below:

As shown above, compounds of the present invention can be formed using palladium catalyzed cross coupling (Suzuki coupling) and ether formation (for example, Williamson synthesis).

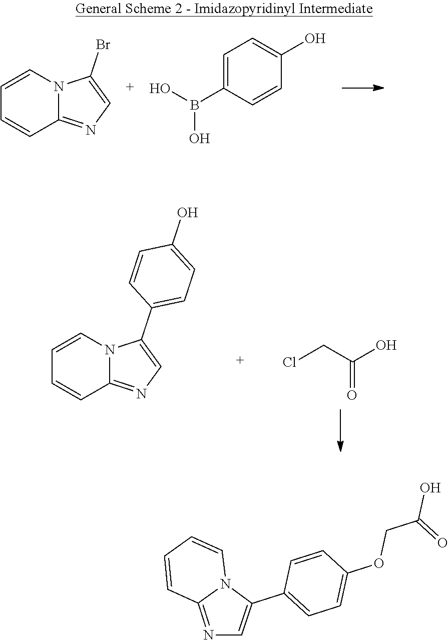 [SMILES: Brc1cnc2ccccn12 ; OB(O)c1ccc(O)cc1 ; Oc1ccc(-c2cnc3ccccn23)cc1 ; O=C(O)CCl ; O=C(O)COc1ccc(-c2cnc3ccccn23)cc1]

As shown above, the intermediate can be formed using palladium catalyzed cross coupling (Suzuki coupling) and ether formation (for example, Williamson synthesis).

### Synthesis of N-(3,4-dimethylphenyl)-2-[4-(tetrazol-1-yl)phenoxy]acetamide (Ex 1)

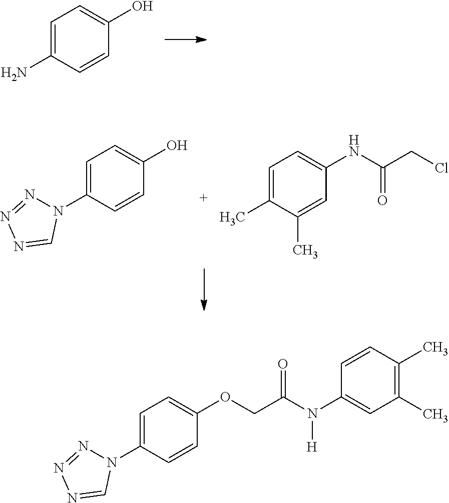 [SMILES: Nc1ccc(O)cc1 ; Oc1ccc(-n2cnnn2)cc1 ; Cc1ccc(NC(=O)CCl)cc1C ; Cc1ccc(NC(=O)COc2ccc(-n3cnnn3)cc2)cc1C]

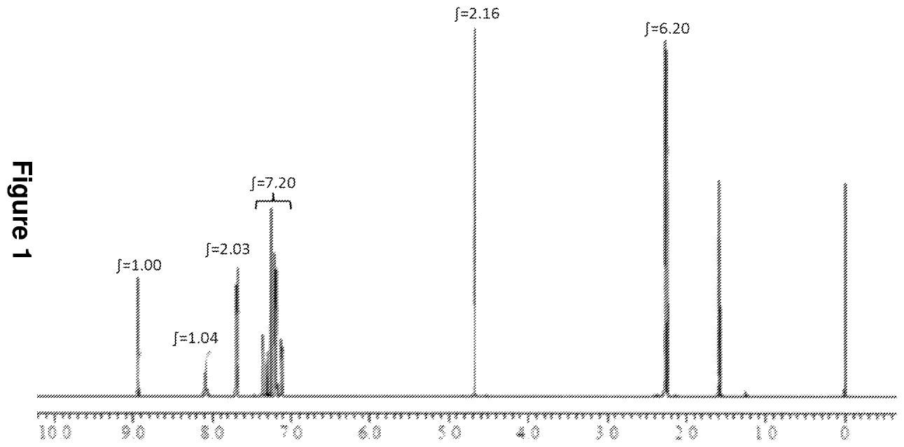

The above compound can be synthesized based on general scheme 1 or general scheme 2 followed by an acid-amine coupling. Alternatively, Ex 1 can be synthesised by reacting arylamine with triethyl orthoformate and sodium azide through the catalyzed reaction with Yb(OTf)₃, and subsequently followed by Williamson ether coupling. The NMR characterisation is as provided in FIG. 1.

### Synthesis of N-(3,4-dimethylphenyl)-2-[4-imidazo[1,2-a]pyridin-3-ylphenoxy]acetamide (Ex 2)

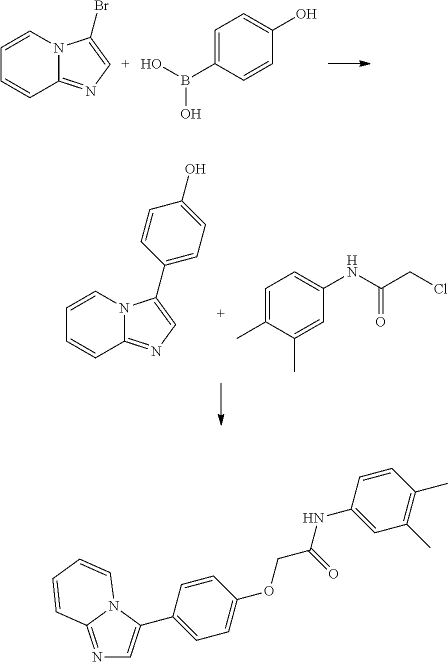 [SMILES: Brc1cnc2ccccn12 ; OB(O)c1ccc(O)cc1 ; Oc1ccc(-c2cnc3ccccn23)cc1 ; Cc1ccc(NC(=O)CCl)cc1C ; Cc1ccc(NC(=O)COc2ccc(-c3cnc4ccccn34)cc2)cc1C]

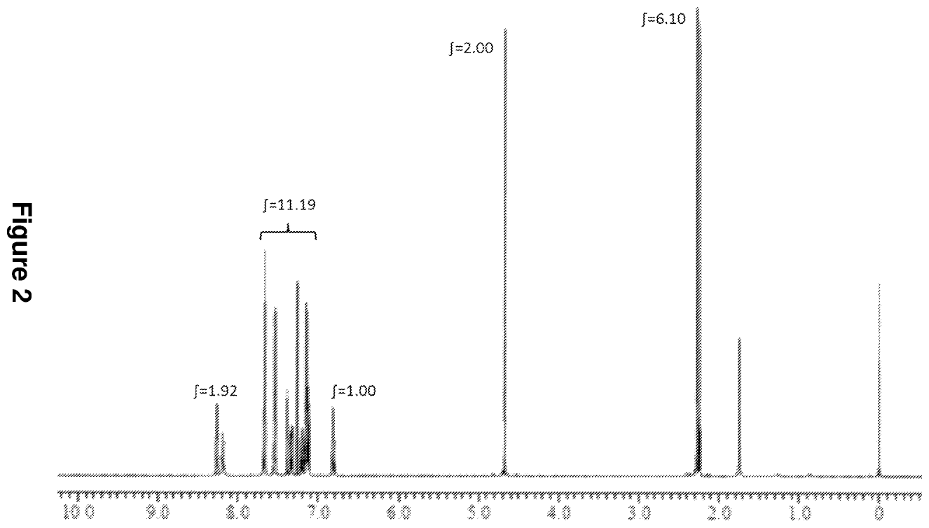

The above compound can be synthesized based on general scheme 1 or general scheme 2. Ex 2 (C₂₃H₂₁N₃O₂; MW 371.43) is a white solid. The NMR characterisation is as provided in FIG. 2. HPLC analysis shows that Ex 2 has a retention time of 9.33 min with a purity of 99.4%.

### Synthesis of N-[4-methyl-3-(trifluoromethyl)phenyl]-2-[4-imidazo[1,2-a]pyridin-3-ylphenoxy]acetamide (Ex 3)

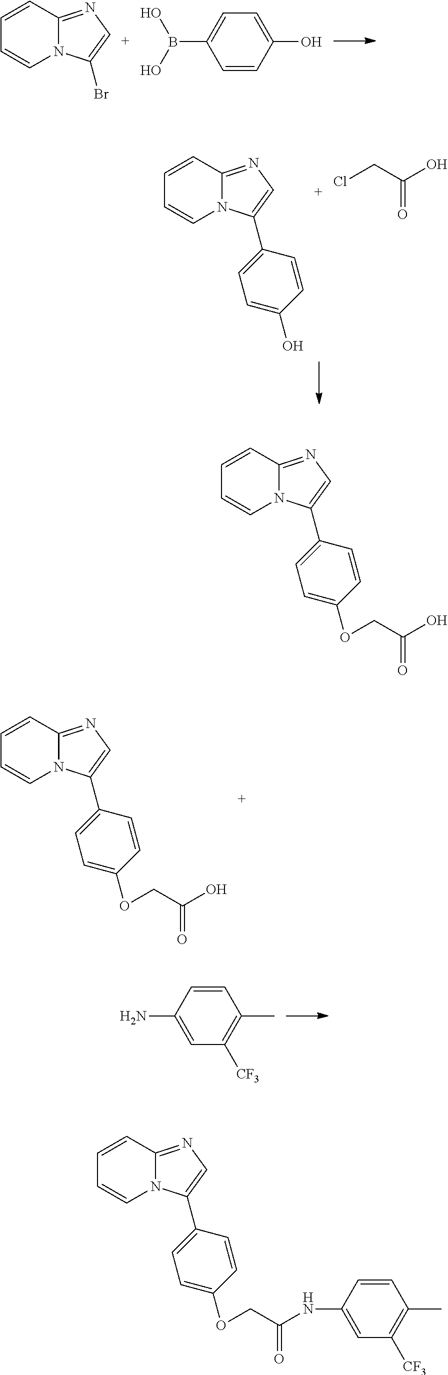

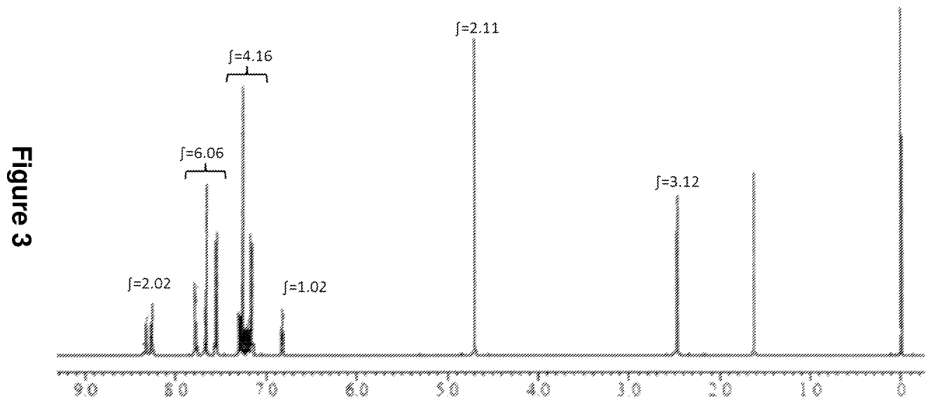

The above compound can be synthesized based on general scheme 1 or general scheme 2 followed by an acid-amine coupling. Ex 3 (C₂₃H₁₈F3N₃O₂; MW 425.40) is a white solid. The NMR characterisation is as provided in FIG. 3. ESIMS m/z [M+H]⁺ 426.62, 427.72, 428.77; [M−H]-424.79, 425.81.

### Biological Data

### Identification of Inhibitors Against (PDGFRβ/TEL-PDGFRβ)WT

A humanized yeast screen was performed. Yeast cells carrying stably integrated GAL-PDGFRβ were first grown in YEP+raff medium and then seeded into fresh YEP+raff+GAL medium to induce expression of human PDGFRβWT. DMSO (final concentration 1%), Gleevac (10 μM) or Ex 1 (10 μM) was added to the growth medium and cell growth was monitored at different time points.

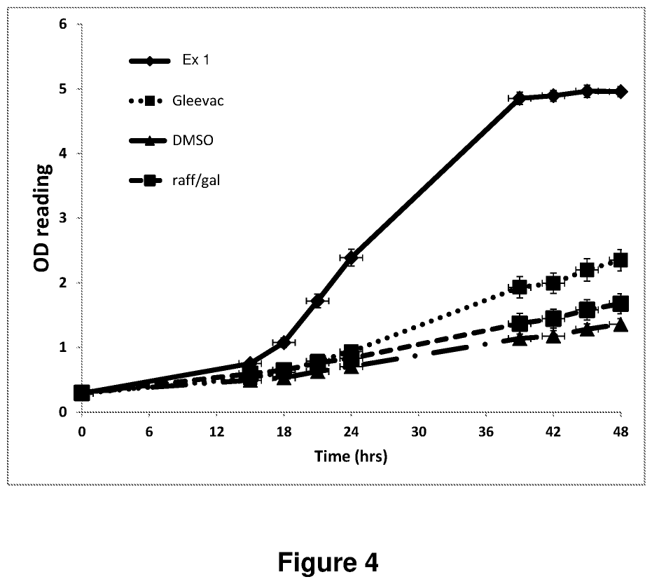

FIG. 4 depicts a graph of OD reading relative to time (hrs) in relation to a compound of the present invention, Gleevac (Imatinib), DMSO and raff/gal (blank). The efficacy of the compound of the present invention was compared to Gleevec (a well-known inhibitor of PDGFR) at 10 μM concentration for its capacity to prevent PDGFR-induced death of humanized yeast cells.

Ex 1 was identified using humanized yeast strain expressing PDGFRβ. Ex 1 allows cell-growth more efficiently compared to DMSO or Gleevec by reversing the growth-detrimental effect of human PDGFRβ.

### Activity in HEK293 Cell Line Model

Human Embryonic kidney cells (HEK293) were constructed to express human PDGFRWT under the control of constitutive CMV promoter. Cells were grown in the presence of 20 ng/ml PDGF (the ligand for the PDGFRβ) to activate the PDGFRβ. Different concentration of Ex 1 were added to study its effect on the PDGFRβ-mediated signaling. Autophosphorylation of PDGFRβ and Shp2 phosphorylation were monitored by Western blotting were used as the markers for PDGFRβ signaling. B Actin was used as an internal control.

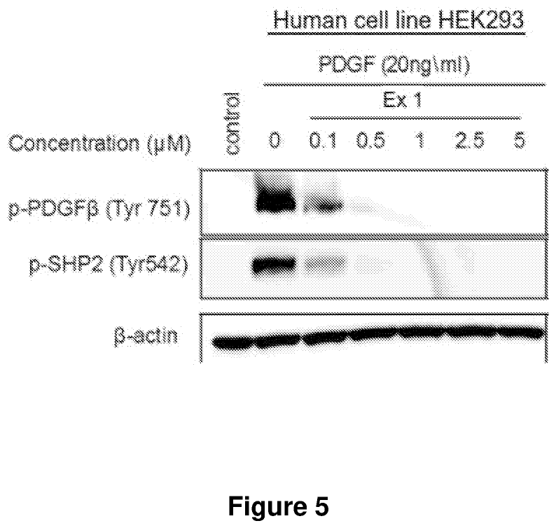

FIG. 5 illustrates that Ex 1 efficiently inhibits the PDGFRWT-mediated signaling.

### Activity in Ba/F3 Cell Line Model

Ba/F3 cells were constructed to express human PDGFRβ under the control of CMV promoter. The PDGFRβ signaling was induced by addition of the ligand PDGF. The effect of Ex 1 and Ex 3 was added to the cell cultures at different concentrations to determine their efficacy to inhibit the PDGFRβ signaling as reflected by the inhibition of PDGFRβ autophosphorylation and the phosphorylation of Shp2, a downstream effector of the PDGFRβ signal pathway.

FIG. 6 illustrates work flow for synthesis of G6 derivatives Effective Concentration-50 (EC₅₀) as determined using Ba/F3 cell.

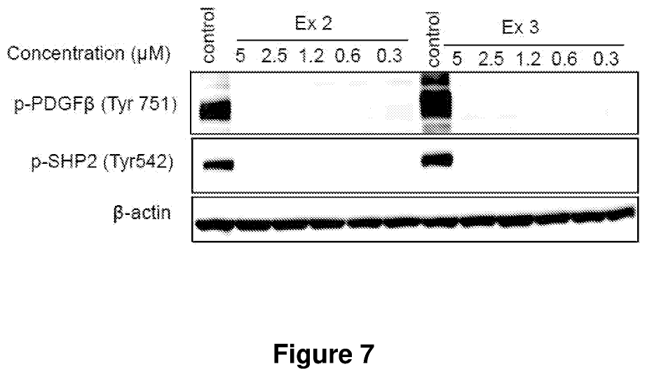

FIG. 7 illustrates a Western-blot analysis result, illustrating the efficacy of compounds of the present invention to inhibit PDGFRβ signaling in BaF3 cells expressing PDGFRβ. Ex 2 and Ex 3 show activities against PDGFRβ-WT in BaF3 cell line model.

### In Vitro Activity and Selectivity Assay

To determine the inhibitory activity of Ex 2 and Ex 3, in vitro kinase assay was performed on PDGFRβ enzyme purified from Sf9 insect cells or E. coli as recombinant GST-fusion proteins or His-tagged proteins. The assay for all protein kinases contained 70 mM HEPES-NaOH pH 7.5, 3 mM MgCl₂, 3 mM MnC₂, 3 μM Na-orthovanadate, 1.2 mM DTT, ATP (variable amounts, corresponding to the apparent ATP-Km of the respective kinase, [γ-33P]-ATP (approx. 9×1005 cpm per well), protein kinase and the peptide substrate. For selectivity assay, Ex 2 and Ex 3 were used against 320 different kinases in a similar kinase assay conditions except that only one concentration of enzyme was used. The selectivity score, according to Karaman et al. (Nat. Biotech. 26, 1, 127-132 (2008)), is a compound concentration-dependent parameter describing the portion of kinases, which are inhibited to more than a predefined degree (e.g. more than 50%), in relation to all tested kinases of the particular project. The selectivity score of the compound at the tested concentrations was calculated for a residual activity <50%, i.e. an inhibition of >50%. The selectivity score for a particular compound at a particular concentration was calculated by using the following formula:

Selectivity Score=(count of data points <50%)/(total number of data points)

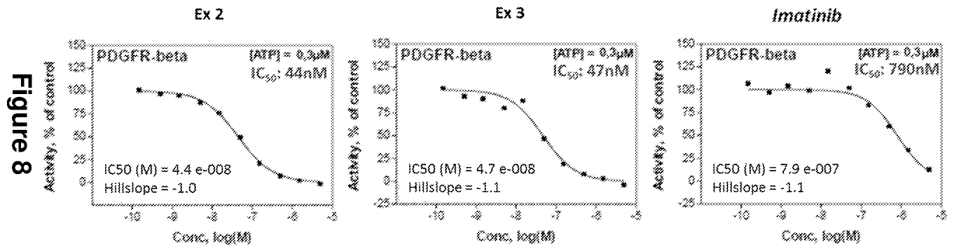

FIG. 8 illustrates the IC₅₀ results of compounds of the present invention using in vitro kinase assay and kinome array. Compounds of the present invention have an IC₅₀ of 44-47 nM against PDGFRβ, compared to Imatinib which has an IC₅₀ of 790 nM. Compounds of the present invention (E×2 and E×3) are also highly selective (>90%) to PDGFRβ, PDGFRα and VEGFR2.

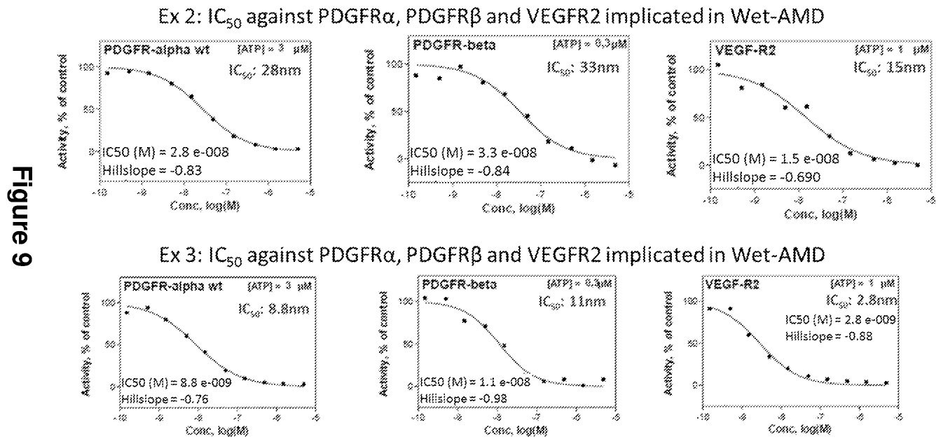

FIG. 9 illustrates IC₅₀ profiles of Ex 2 and Ex 3 against PDGFRα, PDGFR and VEGFR2 using in vitro kinase assay. PDGFRα, PDGFRβ and VEGFR2 receptors are required for angiogenesis and implicated in wet-AMD. The in vitro enzymes assays for IC₅₀ estimation were performed as described in FIG. 8.

### Anti-Angiogenic Activity

### (i) Effect on Capillary Formation In Vitro

Choroid membrane preparations isolated from young mice were cultured in Matrigel. Matrigel containing these preparations were layered with growth medium containing various concentrations of Ex 2 and Ex 3. The ‘blood vessel’ growth was monitored and quantified using a specialized image analysis software.

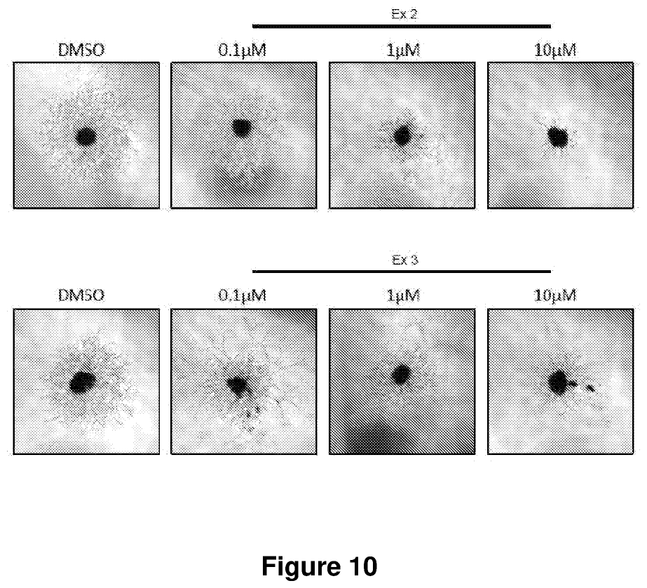

FIG. 10 illustrates the efficacy of the compounds of the present invention in an ex vivo organoid model of angiogenesis. Compounds of the present invention show anti-angiogenesis in organoid model. In particular, Ex 2 showed better anti-angiogenesis efficacy then Ex 3 in this model.

### (ii) Effect on Capillary Formation In Vivo

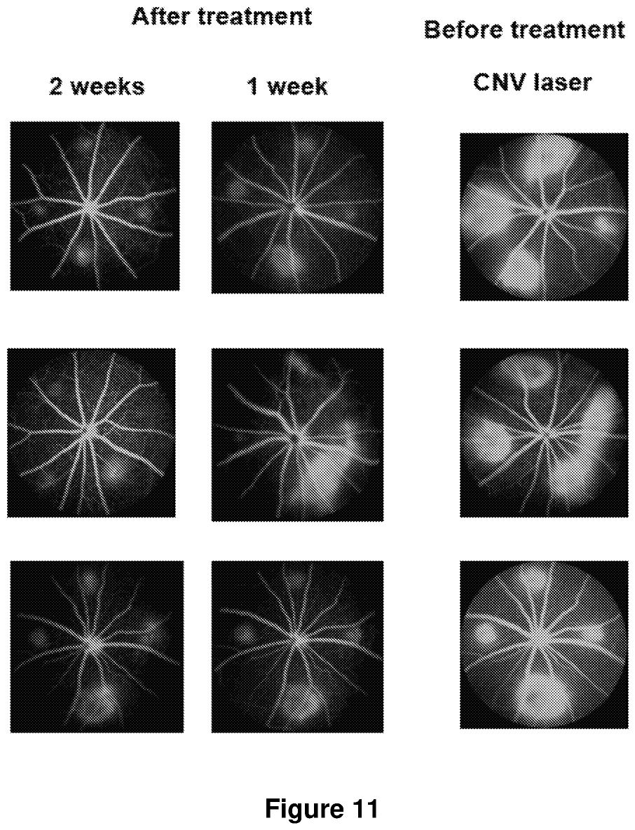

In vivo testing of Ex 3 in CNV (Choroid Neo-Vascularization)-laser mouse model was performed. Eyes of young mice were treated with laser to create choroid damage which results in the leakage of blood vessels (seen as light areas of FIG. 11). In each mouse, one eye was injected with a predetermined amount of Ex 3 while the other eye remained untreated to serve as ‘treatment(−)’ control. Mouse eyes were imaged intermittently for two weeks. The efficacy of the compound Ex 3 is indicated by its ability to prevent the leakage of the blood vessels (reduction or disappearance of the bright green areas).

FIG. 11 illustrates the efficacy of a compound of the present invention in Laser CNV mouse model for wet-AMD.

1. A compound of formula (I) or a salt, solvate, or prodrug thereof: 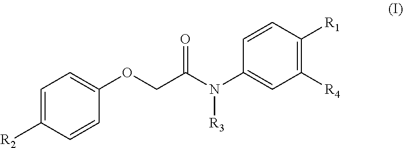
wherein R₁ and R₄ are independently selected from C₁-C₅ alkyl, C₁-C₅ haloalkyl, C₁-C₅ alkenyl, C₁-C₅ haloalkenyl, C₁-C₅ alkynyl, C₁-C₅ haloalkynyl, C₁-C₅ alkoxy, or C₁-C₈ haloalkoxy;
R₂ is an optionally substituted tetrazolyl or an optionally substituted imidazopyridinyl; and
R₃ is H or an optionally substituted alkyl.

2. The compound according to claim 1, wherein at least one of R₁ or R₄ is C₁-C₅ alkyl.

3. The compound according to claim 1, wherein the compound is selected from the following:

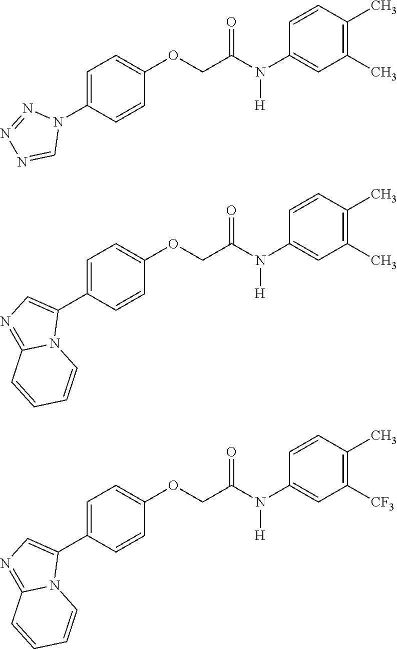 [SMILES: Cc1ccc(NC(=O)COc2ccc(-n3cnnn3)cc2)cc1C ; Cc1ccc(NC(=O)COc2ccc(-c3cnc4ccccn34)cc2)cc1C ; Cc1ccc(NC(=O)COc2ccc(-c3cnc4ccccn34)cc2)cc1C(F)(F)F]

4. A pharmaceutical composition comprising an effective amount of a compound of formula (I) or a pharmaceutically acceptable salt, solvate, or prodrug thereof: 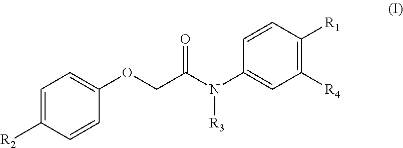
optionally in combination with a pharmaceutically acceptable carrier, excipient, or diluent;
wherein R₁ and R₄ are independently selected from C₁-C₅ alkyl, C₁-C₅ haloalkyl, C₁-C₅ alkenyl, C₁-C₅ haloalkenyl, C₁-C₅ alkynyl C₁-C₅ haloalkynyl, C₁-C₅ alkoxy or C₁-C₅ haloalkoxy;
R₂ is an optionally substituted tetrazolyl or an optionally substituted imidazopyridinyl; and
R₃ is H or an optionally substituted alkyl.

5. The pharmaceutical composition according to claim 4, further comprising an effective amount of a second therapeutic agent.

6. A method for treating macular degeneration (MD) and/or diabetic retinopathy in a patient in need thereof, the method comprising administering to the patient a therapeutically effective amount of a compound of formula (I) or a pharmaceutically acceptable salt, solvate or prodrug thereof: 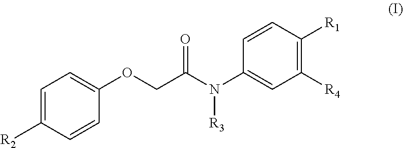
wherein R₁ and R₄ are independently selected from C₁-C₅ alkyl, C₁-C₅ haloalkyl, C₁-C₅ alkenyl, C₁-C₅ haloalkenyl, C₁-C₅ alkynyl, C₁-C₅ haloalkynyl, C₁-C₅ alkoxy, or C₁-C₅ haloalkoxy;
R₂ is an optionally substituted tetrazolyl or an optionally substituted imidazopyridinyl; and
R₃ is H or an optionally substituted alkyl.

7-11. (canceled)

12. The method according to claim 6, wherein the compound of formula (I) or a pharmaceutically acceptable salt, solvate, or prodrug thereof is injected directly to the eye.

13. The method according to claim 6, wherein the compound of formula (I) or a pharmaceutically acceptable salt, solvate, or prodrug thereof is provided on the surface of the eye.

14. The method according to claim 6, wherein the compound of formula (I) or a pharmaceutically acceptable salt, solvate, or prodrug thereof is orally administerable.

15. The compound according to claim 1, wherein R₂ is an optionally substituted heteroaryl, and wherein the optionally substituted group is selected from hydroxyl, acyl, alkyl, alkoxy, alkenyl, alkenyloxy, alkynyl, alkynyloxy, amino, aminoacyl, carboxyl, acylamino, cyano, halogen, 3 nitro, phosphono, sulfo, phosphorylamino, phosphinyl, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents.

16. The pharmaceutical composition according to claim 5, wherein the second therapeutic agent is selected from an angiogenesis inhibitor, a vascular endothelial growth factor (VEGF) inhibitor, a tyrosine kinase inhibitor, photodynamic therapy, laser photocoagulation, and other MD or AMD and/or diabetic retinopathy specific treatments.

17. The pharmaceutical composition according to claim 5, wherein the second therapeutic agent is a VEGF inhibitor selected from ranibizumab, bevacizumab, pegatanib and aflibercept.
